(19) United States
(12) Plant Patent Application Publication
(10) Pub. No.: US 2002/0144321 P1
(43) Pub. Date: Oct. 3, 2002

(54) Blackberry plant named 'Pecos'

(76) Inventor: Carlos D. Fear, Aptos, CA (US)

Correspondence Address: PENNIE AND EDMONDS, 1155 AVENUE OF THE AMERICAS, NEW YORK, NY, 100362711

(21) Appl. No.: 09/772,211

(22) Filed: Jan. 29, 2001

(51) Int. Cl. A01H 5/00

(52) U.S. Cl. PLT/203

(57) ABSTRACT

The present invention relates to a new and distinct cultivar of blackberry plant named ‘Pecos’. The new cultivar is distinguished from other blackberry cultivars by its fruit of excellent fruit firmness and shipping quality. ‘Pecos’ is a thornless midseason cultivar. The new cultivar is distinguished from its seed parent by its earlier season, lower chill requirement, and greater vigor. The new cultivar is distinguished from its pollen parent by having firmer fruit and better fresh market shipping quality.

### 1. BACKGROUND OF THE INVENTION

This invention relates to a new cultivar of blackberry called ‘Pecos’. The new cultivar was developed from hybridization of the patented female selection ‘Navaho’, U.S. Plant Pat. No. 6,679, with the patented male cultivar ‘Loch Ness’, U.S. Plant Pat. No. 6,782. The parents were crossed in Spring 1991 whereafter fruit and seed were collected to produce seedlings for field planting in Watsonville, Calif. in 1991. The new cultivar was selected in July 1993 for its thornless canes and firm, attractive fruit. The cultivar has been asexually propagated, and reproduced true to type plants by in vitro shoot tip culture, root sucker division, and root cuttings.

### 2. SUMMARY OF THE INVENTION

The present invention provides a new and distinct blackberry cultivar named ‘Pecos’. The variety is botanically identified as Rubus L. subgenus Rubus. The new cultivar produces a floricane crop which begins in late June and continues until late August. The new blackberry variety is distinguished from other varieties by a number of characteristics as set forth in Table 1. In particular, the new cultivar is distinguished by its thornless canes, midsummer production, firm fruit and excellent shipping qualities. The fruit of ‘Pecos’ is medium glossy and medium-sized compared to other cultivars. It has a solid black color with minimal postharvest drupelet color reversion.

### 3. COMPARISON TO SIMILAR VARIETIES

The varieties that we believe to be similar to ‘Pecos’ from those known to us are its parent varieties, ‘Loch Ness’ (U.S. Plant Pat. No. 6,782) and ‘Navaho’ (U.S. Plant Pat. No. 6,679). ‘Pecos’ is intermediate between its parents for many characteristics. ‘Pecos’ is particularly characterized by having more vigor and a lower chilling requirement than ‘Navaho’, and having firmer fruit that ‘Loch Ness’. The season of ripening of‘Pecos’ is similar to that of ‘Loch Ness’ and earlier than that of ‘Navaho’.

### 4. BRIEF DESCRIPTION OF THE DRAWINGS

The accompanying photographs show typical specimens of the fruit, leaves and shoot of the new cultivar, in color as nearly true as reasonably possible in color illustrations of this type.

FIG. 1 is a photograph showing a primocane shoot and mature leaf of ‘Pecos’.

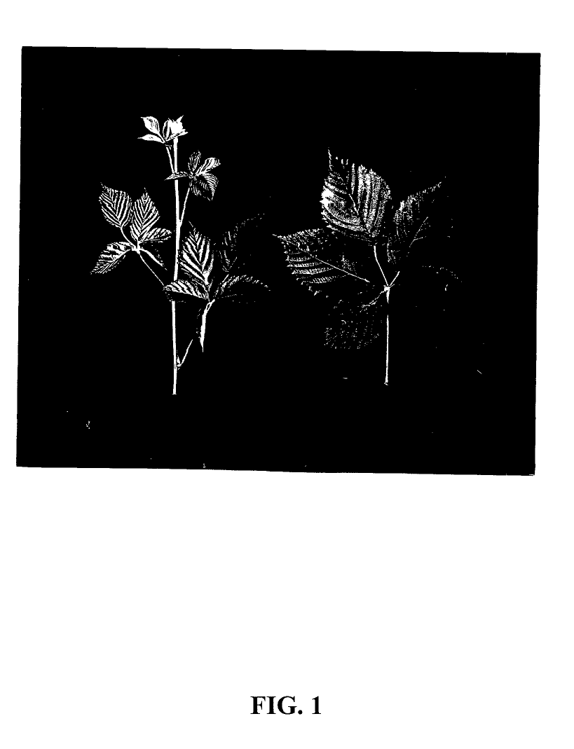

FIG. 2 is a photograph showing a ‘Pecos’ fruiting lateral with fruit in various stages of development.

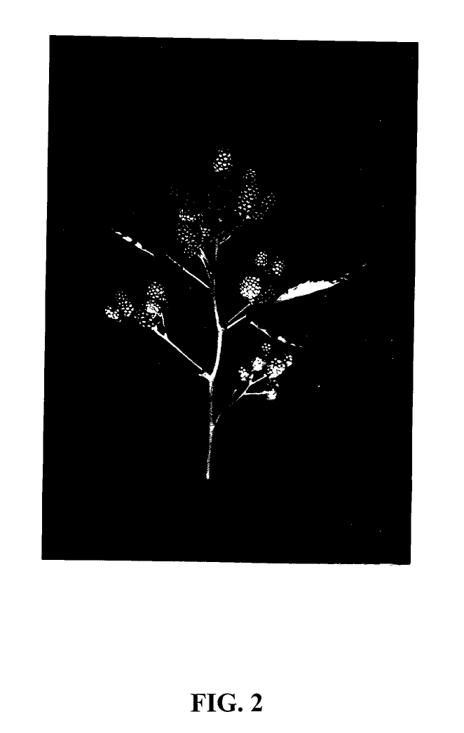

### 5. DESCRIPTION OF THE NEW VARIETY

The following detailed description of the new blackberry cultivar, ‘Pecos’, is based upon recorded observations of plants and fruit grown between 1996 and 2000 in Watsonville, Calif., and is believed to apply to plants of the ‘Pecos’ cultivar grown in similar conditions of soil and climate elsewhere. This description is in accordance with terminology used by the International Union for the Protection of New Varieties of Plants (UPOV). Throughout this specification, color names beginning with a small letter signify that the name of the color, as used in common speech, is aptly descriptive. Color data beginning with a capital letter and followed by an alphanumeric code indicate the most similar color designations as provided by The Royal Horticultural Society (RHS) Colour Chart published by the Royal Horticultural Society of London, England. Color designations, color descriptions, and other phenotypical descriptions may deviate from the stated values and descriptions depending upon variation in environmental, seasonal, climatic and cultural conditions.

### 5.2 CHARACTERISTICS OF THE NEW VARIETY

Table 1 provides information on the plant and fruit characteristics of the new blackberry cultivar ‘Pecos’ compared with characteristics of the unpatented blackberry cultivars ‘Olallie’ and ‘Chester’. Both ‘Olallie’ and ‘Chester’ are currently important cultivars for fresh market shipping, and thus are comparable to the proposed use of the new invention, ‘Pecos’.

The new blackberry cultivar is particularly characterized and distinguished from other cultivars by its fruit of excellent fruit firmness and shipping quality. Flavor of ‘Pecos’ has been rated superior to many other cultivars in sensory tests.

Canes of ‘Pecos’ are thornless and moderately vigorous. Yield of the new cultivar is moderate in comparison with other varieties.

‘Pecos’ is distinguished from its pollen parent, ‘Loch Ness’, by having firmer fruit with better fresh market shipping quality. ‘Pecos’ is distinguished from its seed parent, ‘Navaho’ by its earlier season, lower chill requirement and greater vigor.

TABLE 1
PLANT CHARACTERISTICS OF ‘PECOS’

| | Pecos | Olallie | Chester |
|---|---|---|---|
| GENERAL | | | |
| Vigor | moderate | moderate- | high |
| | | high | |
| Growth habit | semi-upright | trailing | semi-upright |
| Productivity | medium | high | high |
| Self fruitfulness | yes | yes | yes |
| Number of young shoots | few | medium | medium |
| CANES | | | |
| Primocanes | | | |
| Anthocyanin coloration | absent | present | present |
| Spines | absent | present | absent |
| color | — | purple | — |
| attitude of tip | — | horizontal | — |
| texture | — | heavy | — |
| presence and distribution on | absent | present; | absent |
| petioles | | irregularly | |
| | | distributed | |
| density in central third of | — | medium | — |
| shoot | | | |
| Internodal distance (cm)- | 2.9 | 2.6 | 3.1 |
| central third of mature cane | | | |
| Glaucosity on full grown | absent or | weak | weak |
| shoot | very weak | | |
| Strength of full grown shoot | strong | medium | strong |
| Cane cross section | angular to | rounded to | angular to |
| | grooved | angular | grooved |
| LEAVES | | | |
| Relief between veins | medium | medium | medium |
| Number of leaflets | sometimes 3 | usually 3 | usually 5 |
| | sometimes 5 | | |
| Leaf color | medium | medium | light |
| upper side | 137A, 147A | 137A, 137B | 147A |
| underside | 146A, 147B | 147B | 146A |
| Glossiness of upper surface | medium to | medium | dull |
| | glossy | | |
| Leaf cross section | concave | concave-flat | concave |
| Terminal leaflet | | | |
| length (cm) | 11 | 8.9 | 11.1 |
| width (cm) | 8.8 | 7.6 | 9 |
| shape | cordate | cordate | cordate |
| tip | acuminate | acuminate | acuminate |
| base | cordate | cordate | cordate |
| margin | double | double | double |
| | serrate | serrate | serrate |
| Lateral leaflet | | | |
| overlap of lateral leaflets | overlapping | overlapping | overlapping |
| length (cm) | 10.6 | 8.7 | 10.2 |
| width (cm) | 7.4 | 6.1 | 7.1 |
| shape | ovate | ovate | ovate |
| tip | acuminate | acuminate | acute |
| base | acute | acute | acute |
| margin | double | double | serrate |
| | serrate | serrate | |
| Petiole | | | |
| mean length (cm) | 7.9 | 5.3 | 7.9 |
| range | 3.7-12.5 | 3.6-8.7 | 3.9-10.2 |
| pigmentation of upper surface | light reddish | green- | purple |
| | | slightly pink | |
| pigmentation of underside | green | green- | green- |
| | | slightly pink | pinkish |
| Length of stalklet | short | very short | medium |
| Rachis length (cm) between | 3 | 2.8 | 3.1 |
| terminal and adjacent lateral | | | |
| leaflets) | | | |
| Stipule orientation | erect | variable; | erect |
| | | clasping | |
| | | to erect | |
| FLOWERS | | | |
| Time of bud burst | late | early | late |
| Time of beginning of | medium | early | late |
| flowering | | | |
| Flower size | medium- | small to | small to |
| | large | medium | medium |
| Petal size | | | |
| length (mm) | 20.3 | 16.5 | 18.3 |
| width (mm) | 14.7 | 11.7 | 10.9 |
| Anthocyanin color of pedicel | absent | absent | present |
| Intensity of pedicel coloration | — | — | weak |
| Length of pedicel | short | long | short |
| Flower number (third node | 0.95 | 3.6 | 2 |
| from tip of lateral) | | | |
| FRUIT | | | |
| Harvest season | mid | early | mid to late |
| Dimensions | 4.5 | 5.2 | 3.2 |
| weight (g/fruit) | | | |
| Size | medium | medium | small |
| length (cm) | 2.1 | 3.3 | 1.9 |
| width (cm) | 1.8 | 1.4 | 1.9 |
| Fruiting lateral length | short | medium | medium-long |
| (in mid cane) | | | |
| mean number of fruit per | 7.2 | 6.2 | 22.8 |
| lateral | | | |
| range | 4-9 | 3-9 | 17-40 |
| Shape | ovate to | narrow | round to |
| | elliptic; | ovate; much | ovate; as |
| | longer than | longer than | long as |
| | broad | broad | broad |
| Color | black | purple-black | black |
| | | to black | |
| immature | 187A | 178A-183B | 184A |
| maturing | 187A | 187A | 200A-202A |
| mature | 202A | 200A | 202A |
| Firmness | very firm | medium | firm |
| Glossiness | medium | medium- | medium |
| | | strong | |
| Soluble solids | 10.8 | 9.7 | 9.9 |
| Titratable acidity (% as citric | 10.6 | 13.3 | 9.9 |
| acid) (ml of added 0.1 N | | | |
| NaOH to pH 8.1) | | | |
| Number of drupelets per fruit | 60 | 86 | 40 |

### 5.2 NUCLEIC ACID FINGERPRINTING

Distinctive patterns of polymorphism can be detected using a variety of nucleic acid analysis methods. In one non-limiting example, molecular genetic maps can be produced using random amplified polymorphic DNA (RAPD) (Williams et al., 1990, “DNA polymorphisms amplified by arbitrary primers are useful as genetic markers”, Nucleic Acids Res. 18(22):6531-5). Using a variety of oligonucleotide primers, alone or in combination, RAPD analysis of ‘Pecos’, Chester, and Olallie yielded DNA fragment patterns that uniquely distinguish each of these genetically distinct genotypes.

We claim:

1. A new and distinctive cultivar of blackberry plant, substantially as shown and described.
